(19) United States
(12) Patent Application Publication
(10) Pub. No.: US 2026/0191581 A1
(43) Pub. Date: Jul. 9, 2026

(54) MODULAR MULTI-FUNCTIONAL RADIOFREQUENCY THERMOCOAGULATION ELECTRODE

(71) Applicant: Shanghai Brightstone Medical Technology Limited, Shanghai (CN)

(72) Inventor: Xianming Cao, Shanghai (CN)

(21) Appl. No.: 19/422,380

(22) Filed: Dec. 16, 2025

(30) Foreign Application Priority Data
Jan. 7, 2025 (CN) 202510022736.7

(51) Int. Cl. A61B 18/14; A61B 18/00; A61B 90/00

(52) U.S. Cl. CPC A61B 18/1477; A61B 2018/00101; A61B 2018/00107; A61B 2018/00589; A61B 2090/3925; A61B 2090/3937

(57) ABSTRACT

A modular multi-functional radiofrequency (RF) thermocoagulation electrode includes an RF cannula and an RF probe. The RF cannula is composed of a needle tube, an insulating layer, a cannula hub, a lock cap, and a protective sleeve, and is available with multiple head structures for clinical selection. The RF probe is composed of a sheath tube, an electrode filament, a probe hub, a color marker tube, a connecting wire, and a plug. The RF cannula and the RF probe are locked together by securing the cannula hub to the probe hub.

### CROSS-REFERENCE TO RELATED APPLICATION

This application claims priority of Chinese Patent Application No. 202510022736.7, filed on January 7, 2025, the entire contents of which are incorporated herein by reference.

### TECHNICAL FIELD

The present disclosure belongs to the technical field of medical devices, and specifically relates to a modular multi-functional radiofrequency (RF) thermocoagulation electrode.

### BACKGROUND

RF thermocoagulation is a minimally invasive treatment technique. Depending on clinical requirements, medical staff can control the intensity of stimulation or lesion current output from an apparatus and select puncture electrode needles of varying diameters, lengths, and shapes to create selective, specific, and precisely localized lesions. RF thermocoagulation can be used for treating chronic pain, also known as intractable pain, which persists for more than one month, including conditions such as systemic neuralgia, lumbar disc herniation, and myofascial pain syndrome.

Currently in the market, most RF products commercially available for clinical use are supplied as separate components: an RF cannula (or RF needle) and an RF probe (or RF electrode). These components are often sourced from different manufacturers, which leads to a significant positional deviation of a top end of the probe within an inner bore of an RF needle, thereby causing substantial errors in temperature measurement. Furthermore, the RF needles are typically available only in a straight-tube form with a beveled tip similar to an injection needle, with no alternative head forms available. Additionally, a surface of a needle tube is typically spray-coated with an insulating layer extending from about 10 mm behind a needle tip all the way to a needle hub. The above solution has the following drawbacks. (1) The head structure is relatively monolithic and cannot provide more choices for clinical practice. (2) A sharp-tipped straight needle is not suitable for use in areas of the human body dense with blood vessels or nerves. It can easily damage blood vessels or nerves, resulting in significant hemorrhage or loss of sensation and even paralysis. (3) Approaching a target with the sharp-tipped straight needle is relatively difficult. Unless an operator possesses extensive knowledge of human anatomy and considerable experience, allowing for accurate placement in a single puncture under computed tomography (CT) guidance, repeated insertion and withdrawal to adjust the direction are often necessary. This consequently leads to muscle damage and pain for patients, as well as a prolonged procedure time. (4) To ensure the dimensional accuracy of an exposed working segment at a front end of the needle, the spray-coated insulating layer can only be applied after the needle is ground to a sharp point. This process makes the needle tip highly susceptible to damage during the spraying operation, thereby resulting in a low product yield. Therefore, in view of the above problems, it is necessary to provide a new modular multi-functional RF thermocoagulation electrode.

### SUMMARY

The present disclosure provides a modular multi-functional RF thermocoagulation electrode. This modular multi-functional RF thermocoagulation electrode has a modular and flexible structure in which an RF cannula and an RF probe are detachable. A cannula hub incorporates a three-way female conical lock fitting that connects to a male conical lock fitting at a base of a probe sheath tube, allowing the probe to be inserted into an inner bore of the cannula and locked, thereby forming a modular structure. Furthermore, a consistent distance between a top end of the RF probe and a needle tip of a needle tube is maintained, thereby ensuring the temperature control accuracy of the modular multi-functional RF thermocoagulation electrode during ablation or thermocoagulation procedures. Following a combined puncture, anesthesia can be administered through an additional channel on the cannula hub via a connected syringe or extension tube to alleviate pain during treatment. The assembled needle tube offers multiple head structures, including a curved sharp needle, a curved blunt needle, and a curved sharp needle with a side opening, which can help minimize patient tissue damage and pain while avoiding injury to blood vessels and motor nerves. By providing RF cannulas with multiple head structures for clinical selection, surgical trauma and patient discomfort are reduced, complications resulting from damage to blood vessels and motor nerves are prevented, and surgical procedures are simplified. Additionally, operational precision is enhanced, displacement caused by intraoperative instrument exchange is minimized, and both procedural convenience and surgical efficiency are effectively improved. Moreover, spray-coated insulation is replaced by a heat-shrink insulating tube, thereby ensuring production continuity and preventing damage to the RF cannula during transportation or the spraying operation. In summary, the problems identified in the background section are effectively solved.

To solve the above technical problems, the present disclosure employs the following technical solutions.

The present disclosure provides a modular multi-functional RF thermocoagulation electrode, which integrates an RF cannula section, an ultrasound-guided RF probe section, an RF electrode, and an injection port into a unified structure.

The RF thermocoagulation electrode includes an RF cannula and an RF probe. The RF cannula is composed of a needle tube, an insulating tube, a cannula hub, a lock cap, and a protective sleeve; and the RF probe is composed of a sheath tube, an electrode filament, a probe hub, a color marker tube, and a connecting wire.

Further, the RF cannula and the RF probe are locked together by securing the cannula hub to the probe hub.

Further, the cannula hub is configured as a three-way structure; a front end of the cannula hub is fixedly connected to the needle tube, and a rear end of the cannula hub is formed as a female conical lock fitting; the female conical lock fitting is lockingly connected to the probe hub that is equipped with a male conical lock fitting; and a lateral channel of the cannula hub is equipped with a female conical lock fitting configured to connect to a syringe or a high-pressure extension tube, and the lock cap is assembled onto the female conical fitting.

Further, a surface of the needle tube is coated with the insulating tube, with an exposed segment length defined between a needle tip of the needle tube and a top end of the insulating tube. This exposed segment length can be predetermined and altered as clinically required.

Further, the needle tube is configured as one of a straight sharp needle, a curved sharp needle, a curved blunt needle, or a curved sharp needle with a side opening.

Further, a front end of the sheath tube is engraved with a pattern for an ultrasound guidance marker, including reticulated lines, a spiral line, or regularly arranged dimples.

Further, a top end of the sheath tube is configured as a closed hemisphere, and a rear end of the sheath tube is fixedly connected to a center of the male conical lock fitting of the probe hub.

Further, the electrode filament is arranged inside an inner bore of the sheath tube; an outer surface of the electrode filament is coated with an insulating layer, and the sheath tube is insulated internally and externally; a top end of the electrode filament is electrically connected and integrated with a top end of the inner bore of the sheath tube; and a rear end of the electrode filament is welded to the connecting wire, and is sealed within an inner cavity of the probe hub with a potting compound.

Further, the color marker tube is made of an elastic material, and sleeved on a rear end of the probe hub and a front end of the connecting wire, serving to indicate a working length of the electrode and to protect a connection end of the connecting wire.

Further, the connecting wire extends from a rear end of the color marker tube; a tail end of the connecting wire is equipped with an integral plug; and a junction between the connecting wire and the plug is configured as a tapered honeycombed flexible joint to prevent damage to the junction.

Compared with the related art, the present disclosure has the following beneficial effects.

The assembled needle tube offers multiple head structure options, including a curved sharp needle, a curved blunt needle, and a curved sharp needle with a side opening. The curved sharp needle can facilitate directional changes during puncture without requiring substantial withdrawal for redirection, thereby simplifying puncture positioning, reducing procedure time, and minimizing patient trauma and discomfort. The curved blunt needle is suitable for use in areas dense with blood vessels and/or nerves to avoid damage to vascular structures or motor nerves. The curved sharp needle with a side opening is applicable for treatments requiring larger thermocoagulation areas in a target region, serving as an alternative to certain pain treatments that originally require a dual-needle approach.

By providing RF cannulas with multiple head structures for clinical selection, surgical trauma and patient discomfort are reduced, complications resulting from damage to blood vessels and motor nerves are prevented, and surgical procedures are simplified. Additionally, operational precision is enhanced, displacement caused by intraoperative instrument exchange is minimized, and both procedural convenience and surgical efficiency are effectively improved. In particular, an ultrasound-enhancing design is added at the top end of the probe, avoiding the hazards of ionizing radiation to operators and patients.

This modular multi-functional RF thermocoagulation electrode has a modular and flexible structure in which the RF cannula section and the RF probe section are detachable. The cannula hub is configured as a three-way female conical lock fitting, while the probe hub is equipped with a male conical lock fitting, allowing the probe sheath tube to be inserted into the inner bore of the cannula and locked, thereby forming a modular structure. Furthermore, a consistent distance between the top end of the probe sheath tube and the needle tip is maintained, thereby ensuring temperature control accuracy of an RF electrode during ablation or thermocoagulation procedures.

Following a combined puncture, anesthesia can be administered through an additional channel on the cannula hub via a connected syringe or extension tube to alleviate pain during treatment.

Moreover, spray-coated insulation is replaced by a heat-shrink insulating tube, thereby ensuring production continuity and preventing damage to the RF cannula during transportation or the spraying operation.

Naturally, any product embodying the present disclosure does not necessarily need to achieve all of the advantages described above simultaneously.

### BRIEF DESCRIPTION OF THE DRAWINGS

To more clearly illustrate the technical solutions in the embodiments of the present disclosure, the accompanying drawings for describing the embodiments are briefly introduced below. Obviously, the drawings described below are only some embodiments of the present disclosure. For those ordinary skilled in the art, other drawings may be obtained according to the accompanying drawings without creative efforts.

FIG. 1 is an overall structural schematic diagram of a modular multi-functional RF thermocoagulation electrode according to the present disclosure;

FIG. 2 is a schematic diagram showing a structure and positional relationship between a top end of a sheath tube and an electrode filament;

FIG. 3 is an enlarged view of a head structure of a curved sharp RF cannula according to an embodiment;

FIG. 4 is a top view of a structure shown in FIG. 3;

FIG. 5 is an enlarged view of a head structure of a curved blunt RF cannula according to an embodiment;

FIG. 6 is a top view of a structure shown in FIG. 5;

FIG. 7 is an enlarged view of a head structure of a curved sharp RF cannula with a side opening according to an embodiment;

FIG. 8 is a top view of a structure shown in FIG. 7; and

FIG. 9 is a structural diagram of the modular RF electrode of the present disclosure integrated with an RF ablation therapy apparatus.

Reference numerals and denotations thereof:

1-RF cannula; 11-needle tube; 12-insulating tube; 13-cannula hub; 131-female conical lock fitting; 132-female conical fitting; 14-lock cap; 2-RF probe; 21-sheath tube; 211-front end; 22-electrode filament; 23-probe hub; 231-male conical lock fitting; 24-color marker tube; 25-connecting wire; and 251-plug.

### DETAILED DESCRIPTION

The technical solutions in the embodiments of the present disclosure are described below clearly and completely with reference to the accompanying drawings. Obviously, the described embodiments are merely some, rather than all of the embodiments of the present disclosure. Based on the embodiments of the present disclosure, all other embodiments obtained by those ordinary skilled in the art without creative efforts fall within the scope of protection of the present disclosure.

In the description of the present disclosure, it is to be understood that the orientation or positional relations indicated by the terms "head", "end", "side", "rear end", "front end", "top end", etc., are used merely for the ease of describing the present disclosure and simplifying the description, rather than indicating or implying that the component or element referred to need to be in a specific orientation or constructed and operated in a specific orientation, and therefore cannot be interpreted as limiting the present disclosure.

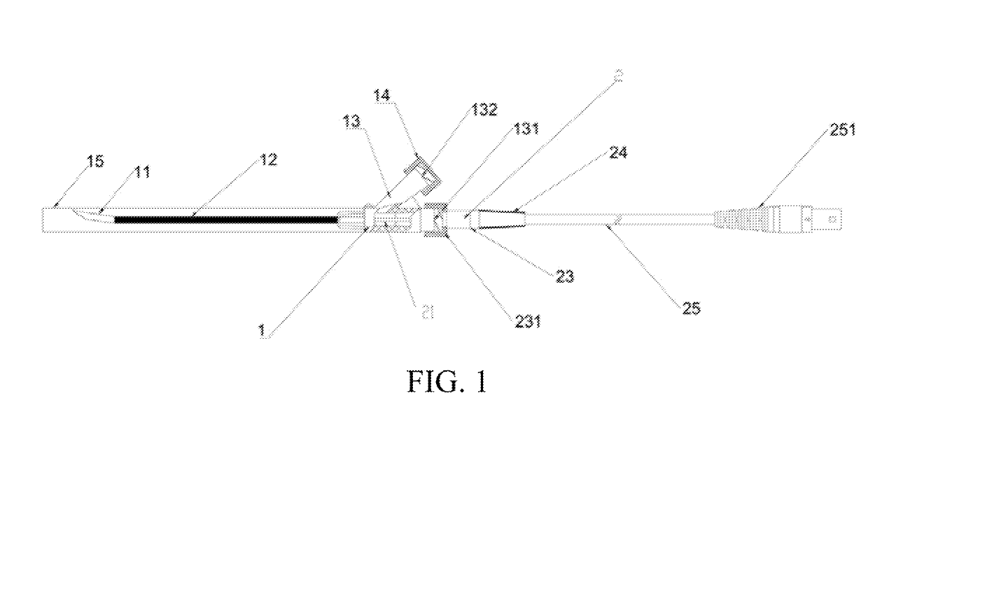

Referring to FIGS. 1-9, the present disclosure provides a modular multi-functional RF thermocoagulation electrode, which integrates an RF cannula 1, an ultrasound-guided RF probe 2, an RF electrode, and an injection port into a unified structure.

The RF thermocoagulation electrode includes the RF cannula 1 and the RF probe 2. The RF cannula 1 is composed of a needle tube 11, an insulating tube 12, a cannula hub 13, a lock cap 14, and a protective sleeve 15; and the RF probe 2 is composed of a sheath tube 21, an electrode filament 22, a probe hub 23, a color marker tube 24, and a connecting wire 25. The insulating tube 12 is a heat-shrink insulating tube, specifically implemented using a medical-grade fluorinated ethylene propylene (FEP) heat-shrink tube.

The RF cannula 1 and the RF probe 2 are locked together by securing the cannula hub 13 to the probe hub 23.

The cannula hub 13 is configured as a three-way structure; and a front end of the cannula hub 13 is fixedly connected to the needle tube 11, and a rear end of the cannula hub 13 is formed as a female conical lock fitting 131. The female conical lock fitting 131 is lockingly connected to the probe hub 23 that is equipped with a male conical lock fitting 231 (i.e., the female conical lock fitting 131 is lockingly connected to the male conical lock fitting 231). A lateral channel of the cannula hub 13 is equipped with a female conical lock fitting 132 configured to connect to a syringe or a high-pressure extension tube, and the lock cap 14 is assembled onto the female conical fitting 132.

A surface of the needle tube 11 is coated with the insulating tube 12, with an exposed segment length defined between a needle tip of the needle tube 11 and a top end of the insulating tube 12. This exposed segment length can be predetermined and altered as clinically required, allowing a predetermined exposed length to be maintained at a head of the needle tube 11 based on therapeutic needs.

The needle tube 11 is configured as one of a straight sharp needle, a curved sharp needle, a curved blunt needle, or a curved sharp needle with a side opening.

A front end 211 of the sheath tube 21 is engraved with a pattern for an ultrasound guidance marker, including reticulated lines, a spiral line, or regularly arranged dimples.

A top end of the sheath tube 21 is configured as a closed hemisphere, and a rear end of the sheath tube 21 is fixedly connected to a center of the male conical lock fitting 231 of the probe hub 23.

The electrode filament 22 is arranged inside an inner bore of the sheath tube 21; an outer surface of the electrode filament 22 is coated with an insulating layer, and the sheath tube 21 is insulated internally and externally. A top end of the electrode filament 22 is electrically connected and integrated with a top end of the inner bore of the sheath tube 21. A rear end of the electrode filament 22 is welded to the connecting wire 25, and is sealed within an inner cavity of the probe hub 23 with a potting compound; and

The color marker tube 24 is made of an elastic material. The color marker tube 24 is sleeved on a rear end of the probe hub 23 and a front end of the connecting wire 25, serving to indicate a working length of the electrode and to protect a connection end of the connecting wire.

The connecting wire 25 extends from a rear end of the color marker tube 24. A tail end of the connecting wire 25 is equipped with an integral plug 251 with the connecting wire 25. A junction between the connecting wire 25 and the plug 251 incorporates a tapered honeycombed flexible joint to prevent damage to the junction.

Referring to FIG. 1, the needle tube 11 is sleeved inside the insulating tube 12 and mounted to an end of the RF cannula 1. A locking connection with the probe hub 23 is achieved through the female conical lock fitting 131 and the male conical lock fitting 231. The RF cannula 1 and the probe hub 23 form a modular and flexible structure with detachable connectivity. The probe hub 23 incorporates a three-way female conical lock fitting that connects to a male conical lock fitting at a base of the probe sheath tube, allowing the RF probe 2 to be inserted into an inner bore of an RF probe section and locked, thereby forming a modular structure. Furthermore, a consistent distance between the needle tip of the needle tube and a top end of the insulating tube 12 is maintained, thereby ensuring the temperature control accuracy of the modular multi-functional RF thermocoagulation electrode during ablation or thermocoagulation procedures. Following a combined puncture, anesthesia can also be administered through an additional channel on the cannula hub via a connected syringe or extension tube to alleviate pain during treatment.

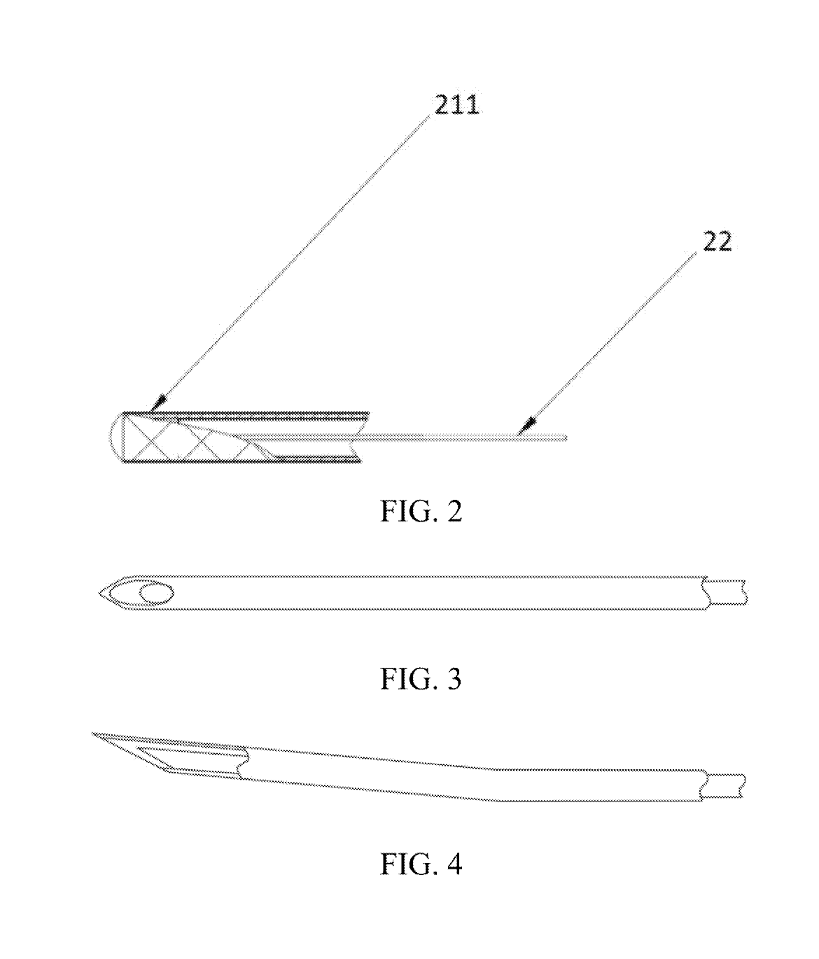

Referring to FIG. 2, it is a schematic diagram showing a positional relationship between the electrode filament 22, the sheath tube 21, and the top end 211 of the sheath tube 21.

Referring to FIGS. 3-4, the curved sharp needle can facilitate directional changes during puncture without requiring substantial withdrawal for redirection, thereby simplifying puncture positioning, reducing procedure time, and minimizing patient trauma and discomfort.

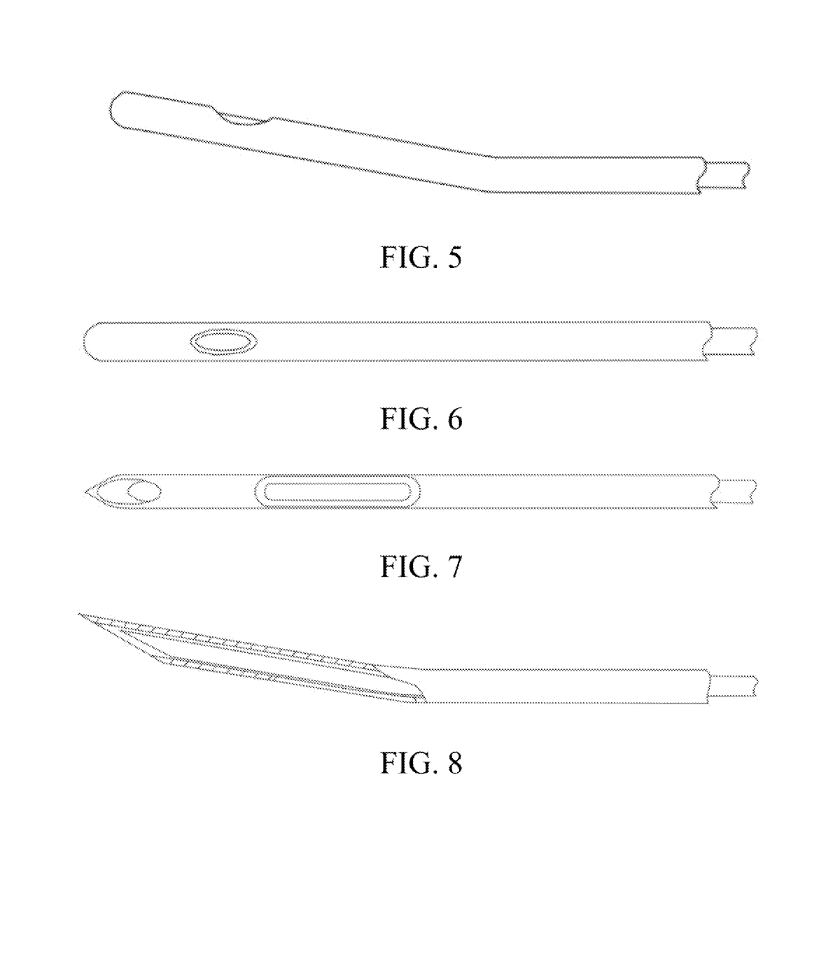

Referring to FIGS. 5-6, the curved blunt needle is suitable for use in areas dense with blood vessels and/or nerves to avoid damage to vascular structures or motor nerves.

Referring to FIGS. 7-8, the curved sharp needle with a side opening is applicable for treatments requiring larger thermocoagulation areas in a target region, serving as an alternative to certain pain treatments that originally require a dual-needle approach.

Spray-coated insulation is replaced by a heat-shrink insulating tube, thereby ensuring production continuity and preventing damage to the RF cannula during transportation or the spraying operation.

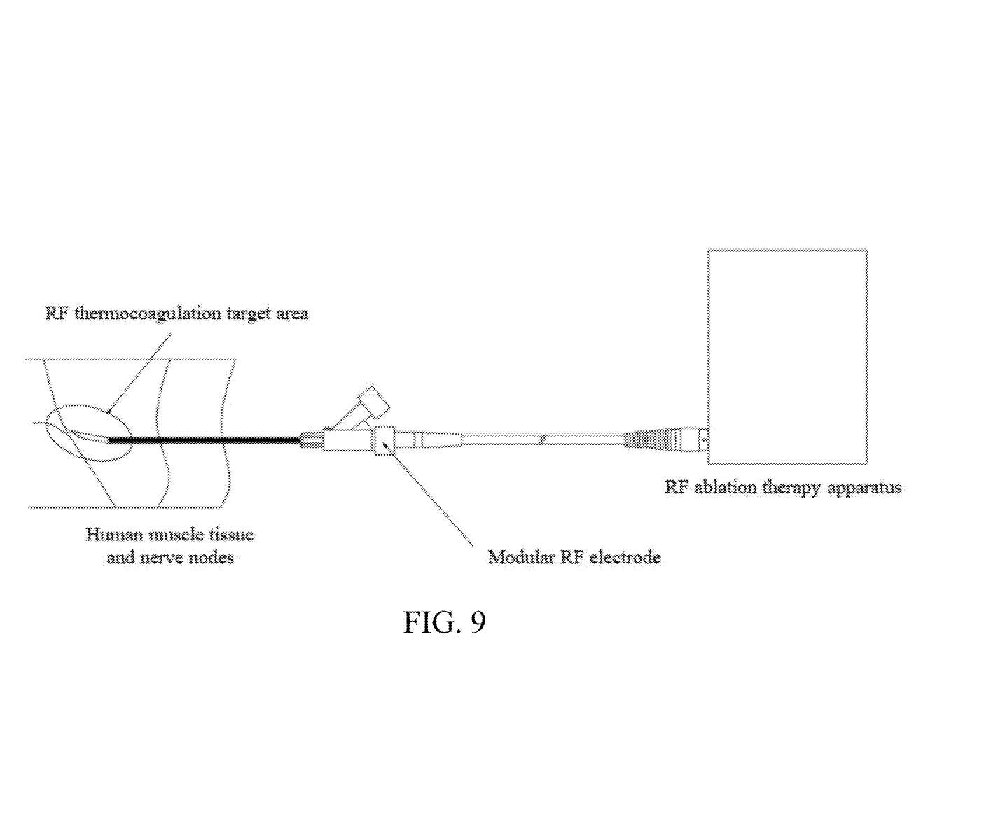

Referring to FIG. 9, during use, the electrode is connected to an RF ablation therapy apparatus. The needle tube 11 at an end of the insulating tube 12 is inserted into a target site within human muscle tissue and nerve nodes, with the needle tube 11 positioned precisely within an RF thermocoagulation target area. The RF ablation therapy apparatus delivers RF current that is conducted through the electrode filament to a distal end of the needle tube 11, where high-temperature thermal energy is generated at an electrode terminal, thereby eliminating the damaged tissue. Once connected to an RF ablation therapy apparatus, the RF thermocoagulation electrode can perform various operations, including temperature monitoring, impedance measurement, electrical stimulation for localization, nerve block, and RF thermocoagulation.

The preferred embodiments of the present disclosure disclosed above are merely for assisting in illustrating the present disclosure. The preferred embodiments are not intended to exhaustively describe all details, nor do they limit the present disclosure to the specific implementations described. Obviously, a plurality of modifications and variations may be made according to the contents of the present specification. These embodiments are selected and specifically described in the specification to better explain the principles and practical applications of the present disclosure, so that a person skilled in the art can well understand and utilize the present disclosure. The present disclosure is limited only by the appended claims and the full scope thereof and equivalents thereof.

1. A modular multi-functional radiofrequency (RF) thermocoagulation electrode, comprising:
an RF cannula; and
an RF probe;
wherein:
the RF cannula is composed of a needle tube, an insulating tube, a cannula hub, a lock cap, and a protective sleeve; and the RF probe is composed of a sheath tube, an electrode filament, a probe hub, a color marker tube, and a connecting wire;
the RF cannula and the RF probe are locked together by securing the cannula hub to the probe hub;
the cannula hub is configured as a three-way structure; a front end of the cannula hub is fixedly connected to the needle tube, and a rear end of the cannula hub is formed as a female conical lock fitting; the female conical lock fitting is lockingly connected to the probe hub that is equipped with a male conical lock fitting; and a lateral channel of the cannula hub is equipped with a female conical fitting configured to connect to a syringe or a high-pressure extension tube, and the lock cap is assembled onto the female conical fitting;
a surface of the needle tube is coated with the insulating tube, with an exposed segment length defined between a needle tip of the needle tube and a top end of the insulating tube;
a front end of the sheath tube is engraved with a pattern for an ultrasound guidance marker, comprising reticulated lines, a spiral line, or regularly arranged dimples;
a top end of the sheath tube is configured as a closed hemisphere, and a rear end of the sheath tube is fixedly connected to a center of the male conical lock fitting of the probe hub;
the electrode filament is arranged inside an inner bore of the sheath tube; an outer surface of the electrode filament is coated with an insulating layer, and the sheath tube is insulated internally and externally; a top end of the electrode filament is electrically connected and integrated with a top end of the inner bore of the sheath tube; and a rear end of the electrode filament is welded to the connecting wire, and is sealed within an inner cavity of the probe hub with a potting compound; and
a consistent distance between a top end of the RF probe and the needle tip of the needle tube is maintained, ensuring precise temperature control of the modular multi-functional RF thermocoagulation electrode during ablation or thermocoagulation procedures.

2. The modular multi-functional RF thermocoagulation electrode according to claim 1, wherein the needle tube is configured as one of a straight sharp needle, a curved sharp needle, a curved blunt needle, or a curved sharp needle with a side opening.

3. The modular multi-functional RF thermocoagulation electrode according to claim 1, wherein the color marker tube is made of an elastic material; and the color marker tube is sleeved on a rear end of the probe hub and a front end of the connecting wire, and configured to indicate a working length of the electrode and to protect a connection end of the connecting wire.

4. The modular multi-functional RF thermocoagulation electrode according to claim 1, wherein the connecting wire extends from a rear end of the color marker tube; a tail end of the connecting wire is equipped with an integral plug with the connecting wire; and a junction between the connecting wire and the plug is configured as a tapered honeycombed flexible joint to prevent damage to the junction.
